(19) United States
(12) Patent Application Publication
(10) Pub. No.: US 2026/0213022 A1
(43) Pub. Date: Jul. 23, 2026

(54) PERIOPERATIVE VULNERABLE BRAIN RISK ASSESSMENT SYSTEM BASED ON MULTIMODAL DATA

(71) Applicant: Fujian Medical University Union Hospital, Fuzhou (CN)

(72) Inventors: Zhongmeng Lai, Fuzhou (CN); Ying Yu, Fuzhou (CN); Peichang Liu, Fuzhou (CN)

(21) Appl. No.: 19/568,961

(22) Filed: Mar. 17, 2026

(51) Int. Cl. G16H 50/30; G16H 10/20; G16H 10/60; G16H 30/40; G16H 50/20

(52) U.S. Cl. CPC G16H 50/30; G16H 10/20; G16H 10/60; G16H 30/40; G16H 50/20

(57) ABSTRACT

A perioperative vulnerable brain risk assessment system based on multimodal data includes: a data acquisition and processing module configured to process multimodal clinical data to construct a subjective feature vector, an objective feature vector, a baseline state vector, and a static brain network atlas; a network atlas reconstruction module configured to generate a perturbation matrix, apply the perturbation matrix to the atlas, and extract an objective physiological latent vector; a vulnerability quotient quantification module configured to calculate a distance between the subjective latent vector and the objective latent vector and generate a vulnerability reserve quotient in combination with a physiological tolerance threshold; a cascading evolution simulation module configured to iteratively update an evolutionary adjacency matrix, and extract a resilience evolution latent vector; and a risk prediction and decision-making module configured to fuse multi-dimensional feature vectors and the quotient, and output a risk probability and target node distribution spatial coordinates.

### TECHNICAL FIELD

The present disclosure relates to the technical field of medical data processing, in particular to a perioperative vulnerable brain risk assessment system based on multimodal data.

### BACKGROUND

Perioperative adverse neurocognitive outcomes are common complications of surgical procedures. Accurate preoperative risk assessment is crucial for clinical early intervention. Routine clinical assessment requires the acquisition of multidimensional data. This initial information typically includes subjective psychological scale scores, objective blood biochemical indicators, and structural or functional magnetic resonance imaging (MRI) images of a brain of a patient. The medical community commonly uses the MRI images to construct a brain network atlas. Brain network analysis is a core method for exploring connection relationships and functional states across various regions of a cerebral cortex.

An existing prognosis risk assessment system usually employs a static feature processing architecture. The system usually directly combines the patient's subjective scale values with objective biochemical test results into a one-dimensional feature vector, and imaging data are extracted and transformed into a resting-state static brain network atlas, where the node features and edge weights of this atlas are fixed in a computational model. A conventional machine learning or deep learning model receives these fused static features, a classification network performs fitting based on past clinical features from a training set, and an assessment model ultimately outputs a risk probability value for the input preoperative slice data.

However, a conventional technical architecture has analytical limitations. A complex compensatory mechanism exists between the subjective emotional stress and the objective physiological functions of the patient. Simple feature concatenation makes it difficult to compute the alignment deviation of heterogeneous data in a deep latent space, and thus the system fails to quantify a decline in the latent physiological reserve of the patient, leading to a potential underestimation of the true vulnerability in high-risk individuals. Surgery is a continuous dynamic stress event accompanied by anesthetic drug impact and traumatic inflammation. A static network model is inadequate for reflecting the energy dissipation phenomenon of local brain region nodes under sustained high stress, thereby missing the temporal dynamic features of network topology cascading failure. Moreover, a front-end data processing network and a back-end risk prediction classifier are forced to be separated into a plurality of independent training stages. Multi-stage independent optimization leads to significant error accumulation, such that it is difficult for the system to achieve end-to-end global parameter iteration. Therefore, the present disclosure provides a perioperative vulnerable brain risk assessment system based on multimodal data to overcome the drawbacks existing in the prior art.

### SUMMARY

In view of the deficiencies in the prior art, the present disclosure provides a perioperative vulnerable brain risk assessment system based on multimodal data, which solves the problems that an existing perioperative brain function risk assessment mechanism mainly relies on single-dimensional clinical indicators or static imaging features, lacks quantitative analysis of an interaction mechanism between subjective psychological states and objective physiological functions of a patient, and is inadequate for effectively simulating a dynamic evolution and cascading failure process of brain network topology under surgical stress conditions, leading to insufficient prediction accuracy for adverse neurocognitive outcomes.

To achieve the above objective, the present disclosure is implemented through the following technical solution: A perioperative vulnerable brain risk assessment system based on multimodal data includes:
a data acquisition and processing module configured to process multimodal clinical data to construct a subjective feature vector, an objective biochemical feature vector, a baseline state vector, and a static brain network atlas;
a network atlas reconstruction module configured to generate a perturbation matrix from the objective biochemical feature vector, apply the perturbation matrix to the static brain network atlas to obtain a reconstructed brain network atlas, and extract an objective physiological latent vector;
a vulnerability quotient quantification module configured to calculate a vulnerability reserve quotient based on a distance between a subjective psychological latent vector projected from the subjective feature vector and the objective physiological latent vector, in combination with a physiological tolerance threshold generated from the baseline state vector;
a cascading evolution simulation module configured to iteratively update an evolutionary adjacency matrix under the control of the vulnerability reserve quotient and a dynamic surgical stress input tensor generated from the baseline state vector, and extract a resilience evolution latent vector; and
a risk prediction and decision-making module configured to fuse the objective physiological latent vector, the subjective psychological latent vector, the resilience evolution latent vector, and the vulnerability reserve quotient to generate a comprehensive state representation vector, and output a risk probability and target node distribution spatial coordinates through network classification.

Preferably, the data acquisition and processing module includes a subjective feature quantification unit, a biochemical feature smoothing unit, and a brain network atlas construction unit, where
the subjective feature quantification unit is configured to perform normalization on acquired subjective psychological scale scores by introducing an anti-zero-division smoothing term, and map scale score values of different dimensions into a unified numerical interval to generate a subjective feature vector;
the biochemical feature smoothing unit is configured to perform a logarithmic transformation with added smoothing parameters on blood biochemical indicator measurements to eliminate extreme value deviations caused by long-tail distributions, combine the transformed blood biochemical indicators to form an objective biochemical feature vector serving as a latent biochemical perturbation source within the system; and
the brain network atlas construction unit is configured to divide region of interest (ROI) nodes based on an automated anatomical labeling brain atlas to form a graph network node set, extract amplitude of low-frequency fluctuation (ALFF) values to form a node feature matrix, and calculate Pearson correlation coefficients for the time series of pairwise nodes to generate a static adjacency matrix according to an atlas connection threshold, thereby constructing the static brain network atlas through encapsulation.

Preferably, the network atlas reconstruction module includes a biochemical perturbation generation unit and an edge weight reconstruction unit, where
the biochemical perturbation generation unit is configured to input a one-dimensional objective biochemical feature vector into a perturbation multi-layer perceptron model configured with a nonlinear activation function, extract a high-dimensional one-dimensional tensor from an output layer after feature dimensionality elevation in a hidden layer, and perform a tensor reshaping operation along a node ordering direction to construct a two-dimensional feature perturbation matrix with a dimension consistent with a dimension of the atlas; and
the edge weight reconstruction unit is configured to introduce a nonlinear gated Hadamard product mechanism containing learnable weights, and uses a nonlinear activation function to forcibly map the perturbation matrix and a bias matrix to generate a global gating mask within a predetermined numerical interval; and
perform an element-wise multiplication operation of elements at corresponding positions between the global gating mask and the static adjacency matrix, and modulate the static edge weights via attenuation gating using adaptively-adjusted disruption weights for different biochemical perturbation features, to generate the reconstructed brain network atlas.

Preferably, the network atlas reconstruction module further includes a latent feature aggregation unit, where
the latent feature aggregation unit is configured to introduce a self-loop operation on the reconstructed adjacency matrix to construct an augmented matrix free of singularity risks and a diagonal degree matrix thereof, fuse node features through multi-layer graph convolution operations, extract a multi-dimensional feature tensor, smoothly compress the multi-dimensional feature tensor along the node dimension into a one-dimensional global representation using a global average pooling layer, and output the objective physiological latent vector.

Preferably, the vulnerability quotient quantification module includes a latent space projection unit, a compensatory tension calculation unit, and a reserve quotient generation unit, where
the latent space projection unit is configured to, using a nonlinear projection network containing a batch normalization layer, project and map the subjective feature vector in a different feature dimension into a unified latent space with a dimension strictly consistent with a dimension of the objective physiological latent vector, to generate the subjective psychological latent vector;
the compensatory tension calculation unit is configured to calculate an aligned squared Euclidean distance between the subjective psychological latent vector and the objective physiological latent vector in the latent space, introduce an adaptive variance penalty smoothing mechanism during calculation to eliminate abnormal feature noise, and digitally quantify a subjective and objective latent decompensatory tension; and
the reserve quotient generation unit is configured to decode the baseline state vector constructed from basic physiological parameters of a patient using a feedforward perceptron network in combination with a soft activation function, generate an individualized physiological tolerance threshold, and when the aligned squared Euclidean distance exceeds the physiological tolerance threshold, output a positive difference using a rectified linear unit as a nonlinear truncation function to generate the vulnerability reserve quotient.

Preferably, the cascading evolution simulation module includes a temporal stress generation unit and a node energy dissipation unit, where
the temporal stress generation unit is configured to, using a feedforward stress decoding network configured with positive constraints, in combination with a maximum simulation time step and a stress amplification factor, map and construct the baseline state vector into a dynamic surgical stress input tensor that monotonically increases over time steps;
the node energy dissipation unit is configured to extract a sum of edge weights of an adjacency matrix from a previous time step as a dynamic effective connectivity of nodes, take the reconstructed adjacency matrix as an initial evolution baseline, and normalize initial internal residual energy of all nodes to a fully loaded compensatory state; and
in an iterative simulation calculation, the node energy dissipation unit is configured to dynamically and controllably attenuate internal residual energy of each brain region node according to a vulnerability sensitivity multiplier modulated by the vulnerability reserve quotient, the dynamic surgical stress input tensor for the corresponding time step, and the dynamic effective connectivity containing an anti-zero-division smoothing term.

Preferably, the cascading evolution simulation module further comprises a topology dynamic update unit, where
the topology dynamic update unit is configured to, using a predetermined physical failure energy threshold as a truncation baseline, and employing a logistic function with a temperature hyperparameter, nonlinearly and smoothly map the internal residual energy of each brain region node into a continuously differentiable node survival soft mask within a predetermined numerical interval, to construct an end-to-end gradient backpropagation path;
perform a matrix outer product operation by extracting a column vector composed of whole-node survival soft masks to construct a global mask matrix, perform a Hadamard product operation with the adjacency matrix from the previous time step to trigger network cascading transfer, and dynamically update the evolutionary adjacency matrix; and
after reaching a predetermined maximum simulation time step, apply a graph readout mechanism to an extracted final evolutionary adjacency matrix, perform average pooling aggregation along a node feature dimension to compress local discrete states, and extract the resilience evolution latent vector containing dynamic collapse temporal information.

Preferably, the risk prediction and decision-making module includes a feature fusion unit and a risk classification unit, where
the feature fusion unit is configured to perform a one-dimensional tensor concatenation operation along a feature axis on the objective physiological latent vector, the subjective psychological latent vector, the resilience evolution latent vector, and the scalar-dimensional vulnerability reserve quotient, to construct a comprehensive state representation vector with aligned feature dimensions; and
the risk classification unit is configured to input the comprehensive state representation vector into a fully connected feedforward classification network configured with a dropout mechanism, decode the comprehensive state representation vector via a normalized exponential function to generate a two-dimensional risk probability vector, and construct a cross-entropy loss function based on historical clinical follow-up data to perform joint optimization and parameter backpropagation updates; and
during an early warning phase, extract the probability of adverse neurocognitive outcomes exceeding an optimal decision threshold dynamically established by a receiver operating characteristic (ROC) curve of a validation set, and trigger a graded clinical warning in a terminal system.

Preferably, the risk prediction and decision-making module further includes a visualization and localization unit, where
the visualization and localization unit is configured to extract a whole-node survival soft mask vector generated at a final simulation time step, perform descending order determination in combination with a topological degree centrality feature value representing a sum of direct physical connection edges in the static brain network atlas, and calculate and extract a predetermined number of target node indices with top-ranking comprehensive risk indices; and
based on a loaded standard medical imaging atlas template, inversely map the extracted target node indices one by one onto a three-dimensional brain atlas coordinate system, and render, anchor and output the target node distribution spatial coordinates on a structural MRI image interface of the patient.

Preferably, the process of performing joint optimization and parameter backpropagation updates by the risk classification unit specifically includes:
the risk classification unit preprocesses historical clinical follow-up data into a two-dimensional one-hot encoding vector to construct a true label vector, and calculates a prediction error in conjunction with the two-dimensional risk probability vector;
the risk classification unit introduces a positive real number anti-overflow smoothing constant into the cross-entropy loss function to prevent a prediction probability from triggering an underlying computational graph crash; and
during the backpropagation process, the risk classification unit updates network weights using an adaptive moment estimation optimizer, and reversely transmits the prediction error in a backpropagation computational graph through a chain rule, thereby driving learnable parameters of a graph convolutional network, a multi-layer perceptron, and the evolutionary adjacency matrix in the preceding network atlas reconstruction module and cascading evolution simulation module to dynamically iterate towards minimizing the prediction error.

The present disclosure provides a perioperative vulnerable brain risk assessment system based on multimodal data. It has the following beneficial effects:
1. By projecting the subjective feature vector and the objective physiological latent vector into the unified latent space and calculating the aligned squared Euclidean distance between the two, the system establishes a data association between a subjective psychological state and objective physiological functions. Combining the individualized physiological tolerance threshold generated from the baseline state vector to calculate the vulnerability reserve quotient overcomes the limitations of conventional assessment relying on single-modal data, and thus individualized and objective quantification of the perioperative brain function compensatory capacity of the patient is achieved.
2. The cascading evolution simulation module of the present disclosure, using the vulnerability reserve quotient and the time-increasing dynamic surgical stress input tensor as the control conditions, dynamically attenuates the internal residual energy of brain region nodes. The logistic function with the temperature hyperparameter is combined to generate the continuously differentiable node survival soft mask, and the evolutionary adjacency matrix is updated with the node survival soft mask. This mechanism transforms the static brain network atlas into a time-driven evolution model, such that the cascading failure features of the network topology can be calculated and captured, and the prediction accuracy for adverse neurocognitive outcome risks can be improved.
3. The present disclosure constructs an end-to-end assessment architecture supporting global joint optimization and provides intuitive spatial localization references. Because the generation process of the node survival soft mask is continuously differentiable, the prediction error calculated by the risk classification unit can be reversely transmitted through the backpropagation computational graph, thereby directly driving parameter iteration in the preceding network atlas reconstruction module and cascading evolution simulation module, and preventing error accumulation caused by multi-stage independent training. By extracting the target node indices and inversely mapping the target node indices onto the three-dimensional brain atlas coordinate system, the system outputs explicit anatomical location coordinates of brain regions for clinical early warning and early intervention.

### BRIEF DESCRIPTION OF THE DRAWINGS

FIG. 1 is an architecture diagram of a system according to the present disclosure.

FIG. 2 is a flowchart of a method according to the present disclosure.

FIG. 3 is a schematic diagram of a cascading evolution simulation module according to the present disclosure.

FIG. 4(a) is a comparison diagram of model risk prediction performance.

FIG. 4(b) is a diagram of three-dimensional brain network topology and target vulnerable node distribution.

Reference numerals in the figures: 100—data acquisition and processing module; 200—network atlas reconstruction module; 300—vulnerability quotient quantification module; 400—cascading evolution simulation module; and 500—risk prediction and decision-making module.

### DETAILED DESCRIPTIONS OF THE EMBODIMENTS

The technical solutions in embodiments of the present disclosure will be clearly and completely described below in combination with the drawings in the specification. It is obvious that the described embodiments are only a part of, rather than all of, the embodiments of the present disclosure. Based on the embodiments in the present disclosure, all other embodiments obtained by those of ordinary skill in the art without making creative efforts should fall within the protection scope of the present disclosure.

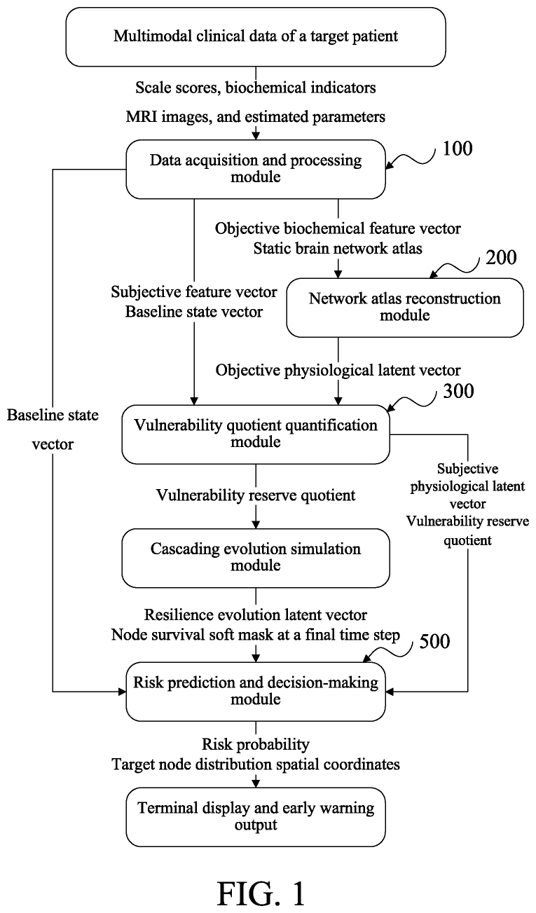

With reference to FIG. 1, FIG. 1 is an architecture diagram of a system according to an embodiment of the present disclosure. The present disclosure provides a perioperative vulnerable brain risk assessment system based on multimodal data, including: a data acquisition and processing module 100, a network atlas reconstruction module 200, a vulnerability quotient quantification module 300, a cascading evolution simulation module 400, and a risk prediction and decision-making module 500. The assessment system is deployed in a medical artificial intelligence inference server, is connected to an information system of a medical institution through a standard data interface, and performs operations for medical heterogeneous data acquisition, structured mapping, graph network computation, and temporal evolution simulation.

The data acquisition and processing module 100 is configured to acquire preoperative subjective psychological scale scores, objective blood biochemical indicators, and resting-state functional MRI images of a target patient, perform normalization on the subjective psychological scale scores to generate a subjective feature vector, perform a logarithmic transformation on the blood biochemical indicators to generate an objective biochemical feature vector, and extract brain region node features and Pearson correlation coefficients between nodes from the functional MRI images to construct a static brain network atlas and a baseline state vector.

The network atlas reconstruction module 200 is communicatively connected to the data acquisition and processing module 100, and is configured to receive the objective biochemical feature vector and the static brain network atlas, map the objective biochemical feature vector into a perturbation matrix with the same dimension as the static brain network atlas using a multi-layer perceptron, apply the perturbation matrix to edge weights of the static brain network atlas through a nonlinear gating mechanism to generate a reconstructed brain network atlas, and perform a graph convolution operation based on the reconstructed brain network atlas to extract an objective physiological latent vector.

The vulnerability quotient quantification module 300 is communicatively connected to the data acquisition and processing module 100 and the network atlas reconstruction module 200 respectively, and is configured to project the subjective feature vector into a predetermined latent space to obtain a subjective psychological latent vector, calculate an aligned squared Euclidean distance between the subjective psychological latent vector and the objective physiological latent vector in the latent space, generate a physiological tolerance threshold according to the baseline state vector, and calculate and output a vulnerability reserve quotient when the aligned squared Euclidean distance exceeds the physiological tolerance threshold.

The cascaded evolution simulation module 400 is communicatively connected to the network atlas reconstruction module 200 and the vulnerability quotient quantification module 300, and is configured to receive the reconstructed brain network atlas and the vulnerability reserve quotient, construct a multi-time-step simulation environment and generate a surgical stress input tensor that increases with time steps, update internal residual energy of each brain region node according to the vulnerability reserve quotient, the surgical stress input tensor, and a dynamic effective connectivity corresponding to the node within each time step, map the internal residual energy to a node survival soft mask using a logistic function, update an evolutionary adjacency matrix by outer product calculation using the node survival soft mask, and extract a resilience evolution latent vector based on a final adjacency matrix after reaching a predetermined simulation time step.

The risk prediction and decision-making module 500 is communicatively connected to the vulnerability quotient quantification module 300 and the cascading evolution simulation module 400, and is configured to receive the objective physiological latent vector, the subjective psychological latent vector, the resilience evolution latent vector, and the vulnerability reserve quotient, concatenate the objective physiological latent vector, the subjective psychological latent vector, the resilience evolution latent vector, and the vulnerability reserve quotient to construct a comprehensive state representation vector, input the comprehensive state representation vector into a feedforward classification network to calculate a probability of the target patient experiencing an adverse neurocognitive outcome, and generate target node distribution spatial coordinates based on the node survival soft mask at a final time step for output to a system interaction terminal.

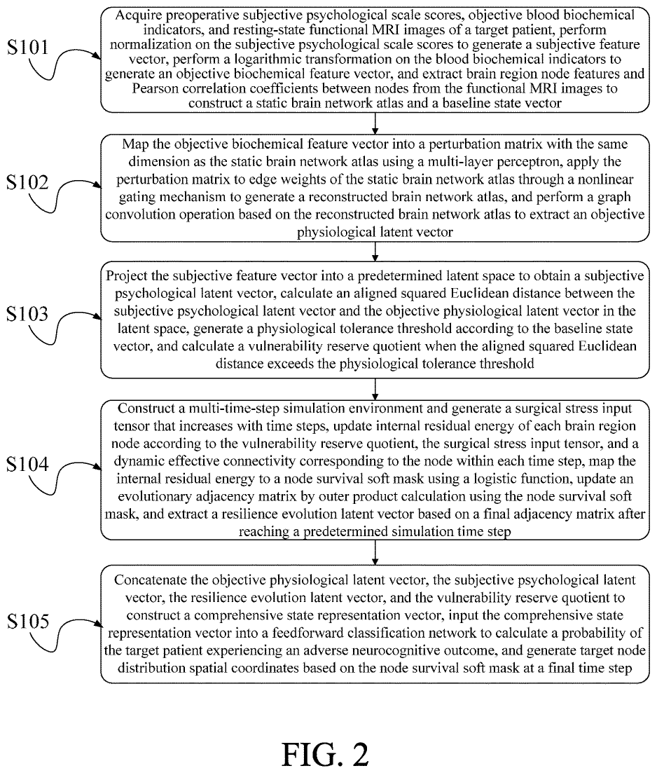

With reference to FIG. 2, FIG. 2 is a flowchart of a method according to an embodiment of the present disclosure. The present disclosure provides a perioperative vulnerable brain risk assessment method based on multimodal data, including the following steps:
S101, preoperative subjective psychological scale scores, objective blood biochemical indicators, and resting-state functional MRI images of a target patient are acquired, normalization is performed on the subjective psychological scale scores to generate a subjective feature vector, a logarithmic transformation is performed on the blood biochemical indicators to generate an objective biochemical feature vector, and brain region node features and Pearson correlation coefficients between nodes are extracted from the functional MRI images to construct a static brain network atlas and a baseline state vector;
S102, the objective biochemical feature vector is mapped into a perturbation matrix with the same dimension as the static brain network atlas using a multi-layer perceptron, the perturbation matrix is applied to edge weights of the static brain network atlas through a nonlinear gating mechanism to generate a reconstructed brain network atlas, and a graph convolution operation is performed based on the reconstructed brain network atlas to extract an objective physiological latent vector;
S103, the subjective feature vector is projected into a predetermined latent space to obtain a subjective psychological latent vector, an aligned squared Euclidean distance between the subjective psychological latent vector and the objective physiological latent vector in the latent space is calculated, a physiological tolerance threshold is generated according to the baseline state vector, and a vulnerability reserve quotient is calculated when the aligned squared Euclidean distance exceeds the physiological tolerance threshold;
S104, a multi-time-step simulation environment is constructed and a surgical stress input tensor that increases with time steps is generated, internal residual energy of each brain region node is updated according to the vulnerability reserve quotient, the surgical stress input tensor, and a dynamic effective connectivity corresponding to the node within each time step, the internal residual energy is mapped to a node survival soft mask using a logistic function, an evolutionary adjacency matrix is updated by outer product calculation using the node survival soft mask, and a resilience evolution latent vector is extracted based on a final adjacency matrix after reaching a predetermined simulation time step; and
S105, the objective physiological latent vector, the subjective psychological latent vector, the resilience evolution latent vector, and the vulnerability reserve quotient are concatenated to construct a comprehensive state representation vector, the comprehensive state representation vector is input into a feedforward classification network to calculate a probability of the target patient experiencing an adverse neurocognitive outcome, and target node distribution spatial coordinates are generated based on the node survival soft mask at a final time step.

The data acquisition and processing module 100 is configured to acquire heterogeneous medical data through a medical information system interface, and perform dimensionality reduction of underlying data features and initialization construction of physical tensors. In this embodiment, the data acquisition and processing module 100 includes a heterogeneous data interface unit, a subjective feature quantification unit, a biochemical feature smoothing unit, and a brain network atlas construction unit.

The heterogeneous data interface unit is configured to establish a data transmission channel with an external medical information system of a medical institution based on Digital Imaging and Communications in Medicine (DICOM) and Health Level Seven International (HL7) standards. Considering objective differences in implementation times of different medical examination items, to ensure immediate consistency of physical meanings during subsequent multi-dimensional feature fusion, the heterogeneous data interface unit predetermines a synchronization time window of 48 hours before surgery, based on a surgery scheduling time, when retrieving perioperative heterogeneous medical data of the target patient. Within this time window, the heterogeneous data interface unit retrieves the subjective psychological scale scores, the blood biochemical indicators, and the resting-state functional MRI images of the target patient from the external medical information system. The subjective psychological scale scores cover a Mini-Mental State Examination (MMSE) score, a Montreal Cognitive Assessment (MoCA) score, and a Clinical Frailty Scale (CFS) score. The blood biochemical indicators include a C-reactive protein concentration and an interleukin-6 concentration related to a neuroinflammation level.

After acquiring the above multi-source data, due to differences in scoring baselines and value ranges of various clinical scales, direct input into a computational network may easily lead to a biased gradient optimization direction. The subjective feature quantification unit receives the subjective psychological scale scores transmitted by the heterogeneous data interface unit, and maps scale scores of different dimensions into a unified numerical interval using a normalization algorithm to generate the subjective feature vector. A calculation formula for a single dimension component of the subjective feature vector is as follows:

$${v}_{scale , i} = \frac{{x}_{i} - {x}_{min , i}}{{x}_{max , i} - {x}_{min , i} + ϵ} ;$$

in the formula, v_{scale,i} represents the i^{th} dimension component of the subjective feature vector; xᵢ represents an actual score value of the target patient on the i^{th} subjective psychological scale; x_{min,i} represents a theoretical minimum score value set for the subjective psychological scale; x_{max,i} represents a theoretical maximum score value set for the subjective psychological scale; and ϵ represents a very small positive real number, for example, a value may be 10⁻⁵, and this parameter serves as a denominator smoothing term to prevent computational crashes caused by division by zero when the input scale score is constant or theoretical extreme values are set abnormally. The subjective feature quantification unit combines all dimension-reduced dimension components into a one-dimensional subjective feature vector, thereby quantitatively characterizing a preoperative subjective psychological compensation potential of the patient in the form of a dense vector.

Different from subjective scale data, concentrations of neurobiochemical markers in human blood may exhibit a long-tail distribution characteristic among different individuals, meaning that inflammatory indicators of a few patients with occult infections may show order-of-magnitude leaps. The biochemical feature smoothing unit performs logarithmic transformation smoothing processing on a concentration measurement of each blood biochemical indicator to eliminate extreme value deviations caused by the long-tail distribution, and generate the objective biochemical feature vector. A calculation formula for dimension components of the objective biochemical feature vector is as follows:

$${v}_{bio , j} = ln ( {y}_{j} + 1 ) ;$$

in the formula, v_{bio,j} represents the j^{th} dimension component of the objective biochemical feature vector; yⱼ represents an actual concentration measurement of the j^{th} blood biochemical indicator of the target patient; constant 1 represents a smoothing parameter used to prevent the system from triggering logarithmic meaningless calculation errors when the concentration measurement is zero. The biochemical feature smoothing unit combines the logarithmically transformed components into a one-dimensional objective biochemical feature vector, which is configured as a core biochemical disturbance source in a subsequent latent physical disruption simulation process.

For high-dimensional medical image data, the brain network atlas construction unit receives the resting-state functional MRI images transmitted by the heterogeneous data interface unit. For basic preprocessing operations such as head motion correction, time layer correction, and spatial normalization of the resting-state functional MRI images, those skilled in the art can implement these operations using existing statistical parametric mapping software, and the processing procedures belong to well-known techniques in the art and are not elaborated here.

After basic preprocessing, to construct an underlying topological structure computable by a graph convolutional network, the brain network atlas construction unit divides the resting-state functional MRI images in the same standard space into a plurality of independent three-dimensional ROI nodes based on an automated anatomical labeling brain atlas, and all ROI nodes constitute a graph network node set. The brain network atlas construction unit extracts ALFF values of each ROI node within a set frequency band range, and combines the ALFF values of all ROI nodes into a two-dimensional node feature matrix which is used to quantitatively represent a baseline intensity of local spontaneous neural activity in each brain region. The brain network atlas construction unit extracts a blood oxygenation level-dependent time series of each ROI node, and obtains a brain network topological connection strength by calculating Pearson correlation coefficients between the time series of pairwise ROI nodes.

During an actual physical atlas construction process, an atlas connection threshold is predetermined in the brain network atlas construction unit. The atlas connection threshold refers to a minimum correlation coefficient boundary for determining existence of a substantive physical connection between two independent ROI nodes, and this value is used to filter weak spurious connections caused by thermal noise of an MRI scanner. As a preferred implementation, a value range of the atlas connection threshold is usually set between 0.15 and 0.25, and its specific value is determined by the system through adaptive optimization according to a principle of network sparsity conservation. The brain network atlas construction unit retains Pearson correlation coefficients greater than the atlas connection threshold as edge weights within the brain network atlas, to generate a static adjacency matrix. The brain network atlas construction unit encapsulates the node feature matrix, the graph network node set, and the static adjacency matrix to form the static brain network atlas.

Boundary conditions of perioperative system evolution are directly constrained by the individual physical function and surgical exogenous load of the patient. The data acquisition and processing module 100 further extracts basic physiological parameters and surgical estimated parameters of the target patient from the medical information system. The basic physiological parameters include an actual age value and a body mass index of the patient. The surgical estimated parameters include an estimated surgery duration and an estimated intraoperative blood loss set under manual intervention. The data acquisition and processing module 100 concatenates and integrates the basic physiological parameters and the surgical estimated parameters to construct and generate a one-dimensional baseline state vector for characterizing the basic physical tolerance ability of the patient.

The network atlas reconstruction module 200 uses medical prior biochemical indicators to perform latent disruption simulation on a physical brain network atlas in a digital space. As a preferred implementation, the network atlas reconstruction module 200 internally includes a biochemical disturbance generation unit, an edge weight reconstruction unit, and a latent feature aggregation unit.

Based on the above medical prior causal correlation, the biochemical disturbance generation unit receives the objective biochemical feature vector and the static brain network atlas transmitted by the data acquisition and processing module 100. To map low-dimensional systemic biochemical features into a high-dimensional topological connection space, the biochemical disturbance generation unit constructs a disturbance multi-layer perceptron model. In this embodiment, the disturbance multi-layer perceptron model includes an input layer, at least two fully connected hidden layers configured with a ReLU nonlinear activation function, and an output layer. The biochemical disturbance generation unit inputs the one-dimensional objective biochemical feature vector into the disturbance multi-layer perceptron model, and after nonlinear feature dimensionality elevation through the fully connected hidden layers, the output layer generates a flattened output of high-dimensional features. To achieve rigorous spatial mapping from a one-dimensional vector to a two-dimensional topological graph, the output layer of the multi-layer perceptron model is precisely configured to contain N² neurons. The biochemical disturbance generation unit extracts an N²-dimensional one-dimensional tensor from this output layer, and performs a tensor reshaping operation along a standard sorting direction of the ROI nodes, converting the tensor into a two-dimensional feature matrix with a dimension consistent with a dimension of the topological atlas, i.e., a perturbation matrix. A generation formula for the perturbation matrix is as follows:

$${M}_{disturb} = Reshape ( {MLP}_{bio} ( {v}_{bio} ) ) ;$$

in the formula, M_{disturb} represents the output perturbation matrix with dimensions N×N, where N represents a total number of ROI nodes in the static brain network atlas; MLP_{bio}(⋅) represents a forward propagation function of the disturbance multi-layer perceptron model; v_{bio} represents the input objective biochemical feature vector; and Reshape(⋅) represents a tensor reshaping function. Each element in the perturbation matrix represents a latent disruption tendency of a neural connection pathway between corresponding pairwise ROI nodes affected by systemic inflammatory infiltration.

Based on the perturbation matrix generated above, the edge weight reconstruction unit performs digital twin reconstruction of the original physical atlas. As a direct physical impact of biochemical indicators on the brain network is usually manifested as a nonlinear inhibition or regulation effect, the edge weight reconstruction unit introduces a nonlinear gated Hadamard product mechanism containing learnable weights. The edge weight reconstruction unit combines the perturbation matrix with the static adjacency matrix in the static brain network atlas, and calculates and generates a reconstructed adjacency matrix. A formula for the reconstructed brain network atlas is as follows:

$${E}_{rew} = {E}_{stat} ⊙ σ ( {W}_{disturb} {M}_{disturb} + {B}_{disturb} ) ;$$

in the formula, E_{rew} represents the reconstructed adjacency matrix, representing an edge weight set of the reconstructed brain network atlas; Eₛₜₐₜ represents the static adjacency matrix; ⊙ represents a Hadamard product operation, i.e., an element-wise multiplication of elements at corresponding positions between two matrices of the same dimension; σ(⋅) represents a Sigmoid nonlinear activation function, used to forcibly map a linear calculation result into a numerical interval between 0 and 1, thereby forming a controlled attenuation gating mechanism; W_{disturb} represents a learnable disturbance weight matrix during model training, with dimensions also set to N×N, which performs standard matrix multiplication with the perturbation matrix here to adaptively adjust relative disruption weights of different biochemical disturbance feature terms on network connections; and B_{disturb} represents a learnable disturbance bias matrix with dimensions N×N.

The numerical matrix generated through σ(⋅) serves as a global gating mask ranging from 0 to 1. When a high degree of neuroinflammation indicated by the blood biochemical indicators leads to an enhanced disturbance response in a specific brain region connection area, a gating coefficient at a corresponding position approaches 0, causing the original static edge weight to be proportionally weakened or even blocked; and conversely, when a corresponding connection is not significantly affected by inflammation, the gating coefficient approaches 1, retaining its original physical connection strength. The edge weight reconstruction unit encapsulates the node feature matrix, the graph network node set, and the reconstructed adjacency matrix to form the reconstructed brain network atlas.

After completing the physical topological reconstruction of the atlas, the system needs to extract a high-dimensional feature representation capable of comprehensively reflecting local brain region spontaneous activity and a whole-brain connection state after reconstruction. The latent feature aggregation unit receives the reconstructed brain network atlas, and performs a graph convolutional network operation based on this. In a specific forward propagation logic of the graph neural network, to prevent gradient dispersion caused by matrix multiplication and matrix singularity problems due to zero diagonal elements, the latent feature aggregation unit introduces a self-loop operation for the reconstructed adjacency matrix E_{rew} to construct an augmented matrix \tilde{E}_{rew} where I represents an N×N identity matrix. Subsequently, the latent feature aggregation unit calculates a diagonal degree matrix \tilde{D} of the augmented matrix, and executes a symmetrically normalized propagation algorithm:

$${H}^{( l + 1 )} = ReLU ( {\tilde{D}}^{- \frac{1}{2}} {\tilde{E}}_{rew} {\tilde{D}}^{- \frac{1}{2}} {H}^{( l )} {W}^{( l )} ) ;$$

in the formula, H^{(l)} represents an input node feature matrix of the l^{th} layer graph convolution, and when, l=0, H⁽⁰⁾ represents an initial node feature matrix of the static brain network atlas; H^{(l+1)} represents a node feature matrix output after calculation by the l^{th} layer graph convolution; W^{(l)} represents a learnable network parameter matrix of the l^{th} layer; ReLU(⋅) represents an activation function; and

$${\tilde{D}}^{- \frac{1}{2}} {\tilde{E}}_{rew} {\tilde{D}}^{- \frac{1}{2}}$$

constitutes a graph Laplacian smoothing operator without a singularity risk.

In this embodiment, the latent feature aggregation unit outputs a node-level multi-dimensional feature tensor after multi-layer graph convolution operations, and compresses a node-level two-dimensional feature tensor into a one-dimensional global representation along a node dimension using a global average pooling layer, to extract the objective physiological latent vector. The objective physiological latent vector not only inherits spatial topological attributes of the objective image, but also deeply integrates micro-inflammatory disruption information mapped by the blood biochemical indicators, thereby providing an objective feature reference system with a physiological basis for subsequent system quantification of the overall latent decompensatory tension of the patient. All learnable matrices in this graph convolutional network and the above multi-layer perceptron are subsequently subjected to end-to-end joint backpropagation training based on clinical samples through a cross-entropy loss function in the subsequent risk prediction and decision-making module 500, to dynamically optimize and obtain optimal parameter configurations.

The vulnerability quotient quantification module 300 quantifies the latent decompensatory tension of the patient through distance calculation in a manifold space. In this embodiment, the vulnerability quotient quantification module 300 is internally divided into a latent space projection unit, a compensatory tension calculation unit, and a reserve quotient generation unit.

The latent space projection unit is communicatively connected to the data acquisition and processing module 100 and the network atlas reconstruction module 200, respectively, and is configured to receive the subjective feature vector. In the multimodal data processing process, as the subjective feature vector extracted from clinical scales and the objective physiological latent vector extracted by the graph convolutional network are in completely different feature dimensions and physical manifold spaces, based on the engineering requirement of the above feature alignment, the latent space projection unit constructs a nonlinear projection network including a fully connected layer and a batch normalization layer. The latent space projection unit projects the one-dimensional subjective feature vector into a predetermined unified latent space using the nonlinear projection network, to generate a subjective psychological latent vector with a dimension strictly consistent with a dimension of the objective physiological latent vector. A projection calculation model is expressed as:

$${h}_{sub} = BN ( {W}_{proj} {v}_{scale} + {b}_{proj} ) ;$$

in the formula, h_{sub} represents the generated subjective psychological latent vector; BN(⋅) represents a batch normalization function, used to prevent feature scale shift during mini-batch forward propagation; Wₚᵣₒⱼ represents a weight matrix of the projection network, whose number of columns is equal to a dimension of the subjective feature vector, and whose number of rows is strictly set to be equal to a dimension d of the objective physiological latent vector, thereby performing precise dimensional mapping; v_{scale} represents the subjective feature vector input from the data acquisition and processing module 100; and bₚᵣₒⱼ represents a bias vector. Through the above spatial transformation operation, the subjective psychological feeling and the objective physiological state are unified into the same mathematical metric space, thereby providing a computable alignment baseline for subsequent tension calculation.

In the unified latent space, a misalignment exists between the apparent psychological stress resistance and the true resilience of the underlying physiological network of the patient, and this misalignment is the latent decompensatory tension. The decompensatory tension calculation unit receives the subjective psychological latent vector and the objective physiological latent vector, and calculates an aligned squared Euclidean distance between the two in the latent space, to digitally quantify the subjective and objective decompensatory tension. To eliminate extreme value interference of potential abnormal feature dimensions on the global distance, the decompensatory tension calculation unit introduces a variance-based feature smoothing mechanism during the calculation process. A specific calculation formula for the aligned squared Euclidean distance is as follows:

$${D}_{align}^{2} = {\sum }_{k = 1}^{d} {( \frac{{h}_{sub , k} - {h}_{obj , k}}{{σ}_{k} + {ϵ}_{norm}} )}^{2} ;$$

in the formula, D_{align}² represents the calculated output aligned squared Euclidean distance; d represents a total number of feature dimensions in the latent space; h_{sub,k} represents the k^{t h} feature component of the subjective psychological latent vector; h_{obj,k} represents the k^{th} feature component of the objective physiological latent vector; σₖ represents a feature standard deviation of the k^{th} dimension in the latent space; during a model training phase, this value takes a statistical standard deviation of a current batch of samples; and during a model inference application phase, this value takes a global moving average standard deviation accumulated during the model training period, used to implement adaptive variance penalty for dimensions, and reduce weights of high-frequency noise dimensions; and ϵₙₒᵣₘ represents a very small positive real constant, as a preferred implementation, a value may be 10⁻⁶, and this constant serves as an anti-zero-division prevention smoothing term, used to prevent computational crashes caused by division by zero when the standard deviation of a certain dimension approaches zero. A larger value of this aligned squared Euclidean distance indicates a more severe deviation between the manifested subjective state and the underlying true physiological network state of the patient, i.e., a higher risk of vulnerability exposure when facing surgical stress.

Clinically, different patients have different physiological tolerance lower limits for the same degree of decompensatory tension. As a preferred implementation, the reserve quotient generation unit introduces the baseline state vector of the patient to dynamically generate an individualized physiological tolerance threshold. The reserve quotient generation unit decodes the baseline state vector using a layer of feedforward perceptron network, and outputs a physiological tolerance threshold in a scalar form in combination with a Softplus activation function ensuring a positive output. A threshold generation formula is as follows:

$${T}_{tol} = Softplus ( {MLP}_{base} ( {v}_{base} ) ) ;$$

in the formula, Tₜₒₗ represents the individualized physiological tolerance threshold; MLP_{base}(⋅) represents a pre-constructed baseline decoding perceptron function, which internally includes a linear mapping layer; Softplus(⋅) represents a smooth nonlinear activation function, defined as Softplus(x)=ln(1+e^{x}), aimed at constraining a network output to be strictly greater than zero, thereby maintaining matching in physical dimension with the above positive squared Euclidean distance; and v_{base} represents a one-dimensional baseline state vector including basic physical parameters such as a patient age, a body mass index, and an estimated surgery duration. The logic of the above calculation lies in that, for patients with advanced age or a heavier estimated surgical load, their underlying risk resistance is weaker. The system will adaptively lower the physiological tolerance threshold for these patients through weight mapping of the multi-layer perceptron, making them more sensitive to the decompensatory tension.

On this basis, the reserve quotient generation unit combines the aligned squared Euclidean distance and the physiological tolerance threshold, performs feature filtering using a nonlinear truncation function, and calculates and outputs a vulnerability reserve quotient. To ensure that the system only responds abnormally to the risk tension exceeding a physiological limit, the reserve quotient generation unit employs a rectified linear unit as the nonlinear truncation function. A final quantification formula for the vulnerability reserve quotient is as follows:

$${Q}_{frail} = ReLU ( {D}_{align}^{2} - {T}_{tol} ) ;$$

in the formula, Q_{frail} represents the vulnerability reserve quotient output by the system; and ReLU(⋅) represents a nonlinear activation function that outputs an original value when an input variable is greater than zero, and outputs zero when the input variable is less than or equal to zero. This formula indicates that when the aligned squared Euclidean distance does not exceed the physiological tolerance threshold, it means the current compensatory deviation is within a safe physiological tolerance range of the patient, and the vulnerability reserve quotient directly outputs zero, and does not exert destructive drive on subsequent network evolution; and when the distance exceeds the physiological tolerance threshold, the truncation function outputs a positive difference value. This positive difference value accurately characterizes a degree of vulnerability overdrawn by the patient when coping with the upcoming surgical stress. The calculated vulnerability reserve quotient is subsequently transmitted to the cascading evolution simulation module 400, and serves as a core driving parameter for controlling dynamic collapse and energy dissipation of the brain network topology.

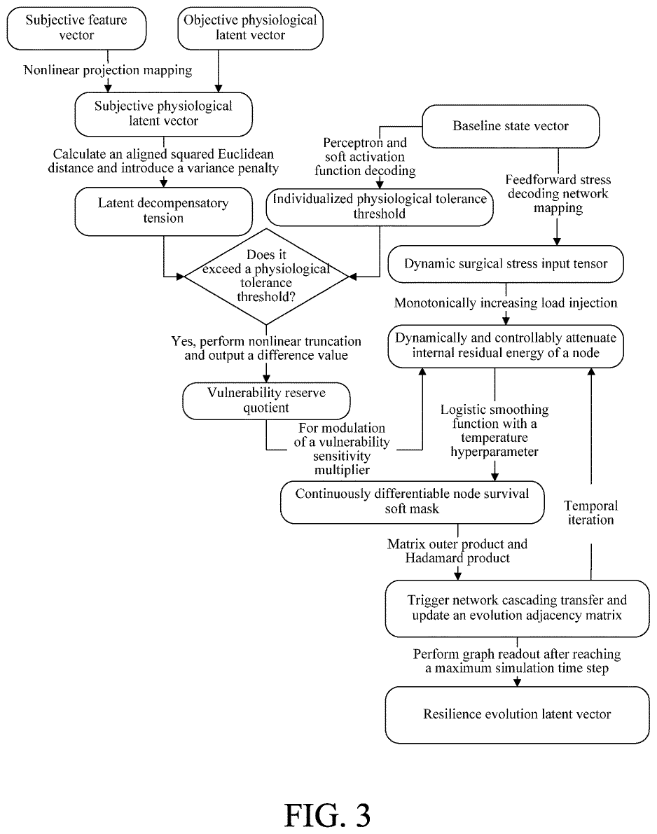

With reference to FIG. 3, the cascading evolution simulation module 400 is configured to simulate a dynamic collapse and compensation process of a brain network of the patient during a surgery in a digital space. A real surgical procedure is a dynamic time-series event that continuously imposes physical and chemical loads on a human body. Based on the above medical prior logic, the cascading evolution simulation module 400 is internally configured with a temporal stress generation unit. This unit receives the baseline state vector, which is integrated from the basic physiological parameters and surgical estimated parameters input by the data acquisition and processing module 100, and constructs a dynamic surgical stress input tensor that increases with time steps in combination with simulation time series parameters.

To precisely map the one-dimensional basic physiological parameters to specific stress loads borne by each brain region node, the temporal stress generation unit constructs a feedforward stress decoding network. In this embodiment, the feedforward stress decoding network includes an input layer, at least one linear hidden layer, and an output layer with a dimension of N, and the output layer is configured with a Softplus nonlinear activation function to ensure that the output stress load values are strictly positive. The temporal stress generation unit constructs the dynamic surgical stress input tensor through this network, and a specific generation formula is as follows:

$${S}_{stress}^{( t )} = {MLP}_{stress} ( {v}_{base} ) \cdot ( 1 + α \frac{t}{{T}_{max}} ) ;$$

in the formula,

$${S}_{stress}^{( t )}$$

represents a dynamic surgical stress input tensor generated at the t^{th} simulation time step, and its dimension is consistent with a total number of ROI nodes in the static brain network atlas; MLPₛₜᵣₑₛₛ(⋅) represents the above feedforward stress decoding network function configured with a positive value constraint, used to map the one-dimensional baseline state vector v_{base} to an initial stress load spatial distribution at a node level; t represents a current simulation time step index; Tₘₐₓ represents a maximum simulation time step length predetermined by the system, used to align with the externally input estimated surgery duration; α represents a stress amplification factor, with a value range usually set between 0.1 and 0.5, and a specific value is determined by external operating condition configuration parameters corresponding to an actual surgery type. This generation logic, through linear or nonlinear increase with time steps, realistically reproduces a phenomenon of a monotonically increasing physiological load caused by the progression of the surgical procedure, accumulation of anesthetic drugs, and increase in blood loss.

With the continuous injection of dynamic surgical stress, nodes within the network atlas will experience energy dissipation. The cascading evolution simulation module 400 is internally configured with a node energy dissipation unit. The node energy dissipation unit receives the reconstructed adjacency matrix output by the network atlas reconstruction module 200 as an initial evolution baseline at a moment zero, and receives the vulnerability reserve quotient output by the vulnerability quotient quantification module 300. In a complex graph topology, nodes with high connectivity can usually effectively disperse exogenous loads through surrounding adjacent nodes, while isolated nodes are prone to physical failure due to local load overload. To initiate time-series calculation, the node energy dissipation unit first uniformly normalizes and sets initial internal residual energy

$${E}_{node , i}^{( 0 )}$$

of all ROI nodes at a moment zero t=0 to a constant 1.0, representing that the nodes are in a fully healthy, fully loaded compensatory state. The node energy dissipation unit extracts a sum of edge weights of various nodes from a previous time step, defines the sum as a node dynamic effective connectivity, and calculates a controlled dissipation evolution equation for the internal residual energy of the node in combination with the vulnerability reserve quotient:

$${E}_{node , i}^{( t )} = {E}_{node , i}^{( t - 1 )} - \frac{{S}_{stress , i}^{( t )} \cdot ( 1 + γ {Q}_{frail} )}{{D}_{eff , i}^{( t - 1 )} + {ϵ}_{deg}} ;$$

in the formula,

$${E}_{node , i}^{( t )}$$

represents internal residual energy of the i^{th} ROI node at the t^{th} time step;

$${E}_{node , i}^{( t - 1 )}$$

represents internal residual energy of the i^{th} ROI node at the t−1^{th} time step;

$${S}_{stress , i}^{( t )}$$

represents a dynamic surgical stress scalar borne by the corresponding node at a current time step; γ represents a predetermined system-level vulnerability sensitivity coefficient, which serves as a weight multiplier to adjust an intervention intensity of the quotient on energy attenuation, with a value range usually set between 0.5 and 2.0; Q_{frail} represents the vulnerability reserve quotient;

$${D}_{eff , i}^{( t - 1 )}$$

represents a dynamic effective connectivity of the i^{th} node at the t−1^{th} time step, calculated as a sum of all edge weights in the i^{th} row of an adjacency matrix from the previous time step; and ϵ_{deg} represents a very small positive real constant, for example, a value can be 10⁻⁵, and this parameter serves as a anti-zero-division smoothing term, used to prevent underlying computational crashes caused when the connectivity drops to zero due to all edges of a certain node breaking.

In a conventional network cascading failure model, once node energy falls below a certain lower limit, the node is directly eliminated. This discrete cliff-like operation blocks gradient calculation in a deep learning framework, such that it is difficult for the model to perform end-to-end joint parameter optimization. The cascading evolution simulation module 400 is internally configured with a topology dynamic update unit. The topology dynamic update unit introduces a logistic function with a temperature hyperparameter to convert the internal residual energy of the node into a continuously differentiable node survival soft mask. A generation formula for the differentiable node survival soft mask is as follows:

$${M}_{surv , i}^{( t )} = \frac{1}{1 + exp ( \frac{{E}_{node , i}^{( t )} - {E}_{th}}{τ} )} ;$$

in the formula,

$${M}_{surv , i}^{( t )}$$

represents a survival soft mask of the t^{th} node at the i^{th} time step, with a value range strictly limited between 0 and 1; Eₜₕ represents a predetermined node physical failure energy threshold, which defines an energy baseline below which the node loses a physiological compensation function, and as a preferred implementation, with a value range set between 0.1 and 0.3; and τ represents a temperature hyperparameter. In this embodiment, a value range of the temperature hyperparameter τ is precisely constrained within an interval of 0.01 to 0.1. When τ approaches 0, this logistic function exhibits an extremely steep curve distribution near the failure threshold, thereby being capable of closely approximating a real physical hard truncation effect, while retaining a weak but complete gradient backpropagation path.

After calculating survival soft mask column vectors for all nodes, the system needs to perform topological transmission of single-point failure states to the global network. The topology dynamic update unit constructs a global survival mask matrix through a matrix outer product operation, and performs a Hadamard product operation with the adjacency matrix from the previous time step to dynamically update the evolution adjacency matrix. A matrix update formula is as follows:

$${E}_{rew}^{( t )} = {E}_{rew}^{( t - 1 )} ⊙ ( {M}_{surv}^{( t )} ⊗ {( {M}_{surv}^{( t )} )}^{T} ) ;$$

in the formula,

$${E}_{rew}^{( t )}$$

represents an updated evolution adjacency matrix at the t^{th} time step;

$${E}_{rew}^{( t - 1 )}$$

represents an updated evolution adjacency matrix at the t−1^{th} time step;

$${M}_{surv}^{( t )}$$

represents a one-dimensional column vector composed of survival soft masks of all nodes; ⊗ represents a matrix outer product operation, where a value of any element (i,j) in the generated two-dimensional matrix is equivalent to a product of the survival soft masks of a node i and a node j;

$${( {M}_{surv}^{( t )} )}^{T}$$

represents a transpose of the column vector; and ⊙ represents a Hadamard product operation. This matrix operation logic ensures that as long as one of two nodes connected by any edge in the network experiences energy depletion, its physical connection weights will exhibit exponential decay, thereby triggering a cascading chain reaction of load transferring to other healthy nodes.

The above state update will be iteratively performed in a fully differentiable computational graph until the predetermined maximum simulation time step Tₘₐₓ is reached. The topology dynamic update unit extracts a final evolution adjacency matrix and internal residual energy states of various nodes output at a final time step. For this final graph topology snapshot, the system employs a graph readout mechanism to perform an average pooling aggregation operation on all nodes along a node feature dimension based on the evolution adjacency matrix. The above aggregation operation smoothly compresses node-level local discrete states, maps these states to a graph-level representation on a whole-brain scale, to precisely extract a resilience evolution latent vector containing complete dynamic collapse temporal information. This resilience evolution latent vector is finally input into a subsequent classification prediction module, and multi-dimensional features therein, under supervision of a global cross-entropy loss function, drive all the above generation networks and perceptron networks to complete end-to-end parameter backpropagation updates.

The risk prediction and decision-making module 500 is configured to integrate the cross-modal representations generated by the preceding modules and output final clinical decision support information. Based on the engineering requirement of the above multi-dimensional feature alignment and evolution simulation, the risk prediction and decision-making module 500 is internally configured with a feature fusion unit. The feature fusion unit respectively receives the objective physiological latent vector output by the network atlas reconstruction module 200, the subjective psychological latent vector and the vulnerability reserve quotient output by the vulnerability quotient quantification module 300, and the resilience evolution latent vector output by the cascading evolution simulation module 400. To ensure that the multimodal data are in the same numerical dimension and feature view before entering a classifier, the feature fusion unit performs a one-dimensional tensor concatenation operation on the above data along a feature dimension, to construct a comprehensive state representation vector. A calculation formula for tensor concatenation is expressed as:

$${V}_{comp} = Concat ( {h}_{obj} , {h}_{sub} , {h}_{evo} , {Q}_{frail} ) ;$$

in the formula, V_{comp} represents the generated comprehensive state representation vector, with a total dimension length Dₜₒₜₐₗ strictly equal to an arithmetic sum of an objective physiological latent vector dimension d_{obj}, a subjective psychological latent vector dimension d_{sub}, a resilience evolution latent vector dimension dₑᵥₒ, and a vulnerability reserve quotient scalar dimension l; Concat(⋅) represents a tensor concatenation function along a specified feature axis; h_{obj} represents the objective physiological latent vector; h_{sub} represents the subjective psychological latent vector; hₑᵥₒ represents the resilience evolution latent vector; and Q_{frail} represents the vulnerability reserve quotient in a scalar form.

After obtaining the global feature representation, the system needs to decode the global feature representation nonlinearly into a clinical risk probability with a practical business meaning. The risk prediction and decision-making module 500 is internally configured with a risk classification unit, which receives the comprehensive state representation vector using a fully connected feedforward classification network. In this embodiment, the fully connected feedforward classification network includes an input layer, at least two hidden layers configured with a ReLU activation function and a Dropout mechanism, and an output layer with a dimension of 2. The Dropout mechanism in the hidden layers can randomly discard some neurons, and is configured to suppress model overfitting phenomena that are very likely to occur in high-dimensional feature fusion scenarios. The risk classification unit, through a final layer of this network in combination with a Softmax normalization function, outputs a binary classification probability distribution regarding occurrence or non-occurrence of an adverse neurocognitive outcome. A risk probability generation formula is as follows:

$${P}_{risk} = Soft max ( {W}_{out} \cdot Re LU ( {W}_{hid} {V}_{comp} + {b}_{hid} ) + {b}_{out} ) ;$$

in the formula, Pᵣᵢₛₖ represents an output two-dimensional risk probability vector, whose two elements respectively represent predicted probabilities of the patient experiencing and not experiencing an adverse neurocognitive outcome post-surgery, and a sum of the two is 1; W_{hid} and b_{hid} respectively represent a learnable weight matrix and a bias vector of a hidden layer; ReLU(⋅) represents a nonlinear activation function of the hidden layer; Wₒᵤₜ and bₒᵤₜ respectively represent a weight matrix and a bias vector of an output layer; and Softmax(⋅) represents an exponential normalization function. A higher predicted probability indicates a more significant risk of functional cognitive impairment caused by collapse of the underlying physiological network of the patient after undergoing a digital intraoperative stress simulation. In actual medical early warning applications, the system does not blindly use 0.5 as a default early warning trigger line. Instead, the system pre-calculates a maximum Youden index based on an ROC curve of a validation set sample, thereby dynamically establishing an optimal decision threshold Pₜₕ. When the extracted probability of adverse outcome occurrence is greater than the dynamic threshold, the system terminal will trigger a graded clinical early warning.

For a neural network model with the above learnable parameters, the system employs an end-to-end joint training mechanism for parameter optimization. During a model construction and training phase, the risk classification unit constructs a true label vector based on externally input historical clinical follow-up validation data, and calculates a prediction error using a cross-entropy loss function with a smoothing term. A specific formula for a joint training loss function is as follows:

$${L}_{CE} = - \frac{1}{M} {\sum }_{m = 1}^{M} {\sum }_{c = 1}^{2} {y}_{m , c} log ( {p}_{m , c} + ϵ ) ;$$

in the formula, L_{CE} represents a global cross-entropy loss function value; M represents a total number of samples in a current training batch; and y_{m,c} represents a true label value of the m^{th} sample in the c^{th} category, and this label originates from clinically followed-up diagnostic gold standard data after desensitization processing. To satisfy matrix dimension alignment requirements for two-dimensional output cross-entropy, the system preprocesses an original scalar label into a two-dimensional one-hot encoding vector before inputting data into the loss function. In this embodiment, when an adverse neurocognitive outcome actually occurs clinically, a corresponding label vector is encoded as [0, 1]; and conversely, when the adverse neurocognitive outcome does not occur, the corresponding label vector is encoded as [1, 0]. P_{m,c} represents a probability value predicted by the classification network that the sample belongs to the c^{th} category; ϵ represents a very small positive real number anti-overflow smoothing constant, with a value range usually set between 10⁻⁷ and 10⁻⁵, aimed at avoiding triggering an underlying computational graph crash when a predicted probability P_{m,c} approaches zero.

During a backpropagation process, the system preferably employs an adaptive moment estimation optimizer to update network weights. To balance early exploration efficiency and later convergence stability, an initial learning rate of the optimizer is set between 10⁻⁴ and 10⁻³. This calculated error will be reversely transmitted through a chain rule in a backpropagation computational graph to all preceding graph convolutions, multi-layer perceptrons, and evolution adjacency matrices, thereby driving parameter matrices of each unit to dynamically iterate towards a direction of minimizing the prediction error. The risk prediction and decision-making module 500 is further internally configured with a visualization and localization unit. To avoid local computational noise interference that may be introduced by relying solely on a single extreme value, the visualization and localization unit extracts a whole-node survival soft mask vector generated by the cascading evolution simulation module 400 at a moment of a predetermined maximum simulation time step, and performs multi-dimensional weighted determination to extract targeted vulnerable nodes in combination with a topological degree centrality of each node in the static brain network atlas. A calculation formula for a comprehensive risk index is as follows:

$${I}_{danger , k} = ( 1 - {M}_{surv , k}^{( {T}_{max} )} ) \cdot {C}_{deg , k} ;$$

in the formula, I_{danger,k} represents a comprehensive risk index for the k^{th} node;

$${M}_{surv , k}^{( {T}_{max} )}$$

represents a survival soft mask value of the node at a final simulation time step; C_{deg,k} represents a degree centrality feature value of the node in an initial static brain network atlas, representing a total number of directly physically connected edges of the node.

By sorting the comprehensive risk indices of all nodes in a descending order, the visualization and localization unit extracts 3 to 5 node indices of a predetermined quantity with the largest values. On this basis, the visualization and localization unit, according to a pre-loaded standard medical image atlas template, inversely maps the extracted topological node indices one by one into a three-dimensional brain atlas, to generate targeted vulnerable node distribution spatial coordinates. By combining with a medical image display interface of a hospital terminal, high-risk failing brain regions are anchored on real structural MRI images of the patient in forms such as highlighting rendering. For pixel-level matching and voxel rendering techniques based on a standard medical image coordinate system, those skilled in the art can implement these techniques using open-source image processing libraries, and specific projection algorithms belong to well-known techniques in the art and are not elaborated here.

To understand the technical solutions of the present disclosure more intuitively, the following description is given in conjunction with a specific application scenario.

To further verify robustness and prediction accuracy of the above medical informatics architecture in a real clinical scenario, and to demonstrate in detail a complete data flow link from multimodal feature fusion to decision output, this embodiment constructs a dedicated offline retrospective experimental validation platform. Considering requirements of high-concurrency tensor computation for stable operation of underlying hardware, model training and inference are deployed in a standard constant-temperature and constant-humidity data center computer room. An ambient temperature is strictly controlled at 22±2° C., and a relative humidity is maintained in a range of 45% to 55%, thereby ensuring that a deployed graphics processing unit cluster does not trigger a thermal throttling protection mechanism during a long-term end-to-end joint training process.

In a specific dataset construction link, the system retrieves multimodal historical electronic medical record data of 500 elderly patients (with an age range between 65 and 85 years) undergoing an elective major surgery under general anesthesia. The dataset internally contains patients' preoperative resting-state functional MRI data, Hospital Anxiety and Depression Scale scores, basic physical parameters such as estimated surgery durations, and gold standard labels of neurocognitive outcomes confirmed by clinical follow-up post-surgery. Among them, there are 120 positive samples where an adverse neurocognitive outcome occurred, and 380 negative samples where the adverse neurocognitive outcome did not occur. Based on the above multi-source heterogeneous data, after performing strict desensitization and image registration preprocessing, the system divides brain regions into 90 independent ROI nodes, and accordingly extracts an objective physiological latent vector in an initial state.

In a vulnerability quotient quantification phase, the system inputs extracted one-dimensional subjective feature vectors such as scale scores into a latent space projection unit configured with a batch normalization layer. The latent space projection unit, using a projection network weight matrix with the number of columns equal to a scale dimension and the number of rows equal to 64, precisely maps these subjective feature vectors into a subjective psychological latent vector with a dimension strictly consistent with a dimension of the objective physiological latent vector. By calculating an aligned squared Euclidean distance between the above two sets of latent vectors in a 64-dimensional manifold space, and in combination with an individualized physiological tolerance threshold generated based on a patient age and a body mass index, the system outputs a vulnerability reserve quotient greater than zero using a truncated rectified linear unit. This quotient intuitively reflects a severe hidden decompensation risk in an underlying neural network of some patients with normal apparent physiological indicators.

Along with dynamic injection of a surgical stress tensor, the cascading evolution simulation module 400 initiates time-series iterative simulation. The system sets a maximum simulation time step to 100 steps to align with an average real surgery load cycle of two hours. Within each time step, internal residual energy of a node is driven by dual consumption of external stress and the vulnerability reserve quotient, and a decay rate of the residual energy is adaptively buffered by a dynamic effective connectivity of the node. When energy of some isolated nodes approaches a physical failure energy threshold set to 0.2, a logistic function with a temperature hyperparameter set to 0.05 smoothly compresses survival soft masks of these nodes to approach zero. This local failure is rapidly propagated to a global evolution adjacency matrix through matrix outer product and Hadamard product operations, thereby triggering a cascading transfer of load to surrounding healthy brain regions. After reaching the 100th time step, the system uses a graph readout mechanism to extract a resilience evolution latent vector containing a whole-brain dynamic collapse topology snapshot.

Entering a risk prediction and decision-making stage, the feature fusion unit concatenates the objective physiological latent vector, the subjective psychological latent vector, the resilience evolution latent vector, and the vulnerability reserve quotient along a feature axis as a one-dimensional tensor, to construct a comprehensive state representation vector with a total dimension of 193. This high-dimensional representation is sent into a fully connected feedforward classification network configured with a Dropout mechanism, and decoded into a two-dimensional risk probability via a Softmax layer. During a model training phase, the system drives all system parameters to complete end-to-end convergence at an initial learning rate of 10-4 using an adaptive moment estimation optimizer, in combination with a cross-entropy loss function set with a very small positive real number anti-overflow smoothing constant.

An experimental environment simultaneously runs two conventional baseline solutions for effect comparison. A first solution is a traditional logistic regression assessment solution relying only on clinical scales and physical indicators. A second solution is a static graph convolutional network solution stripped of a dynamic cascading failure mechanism.

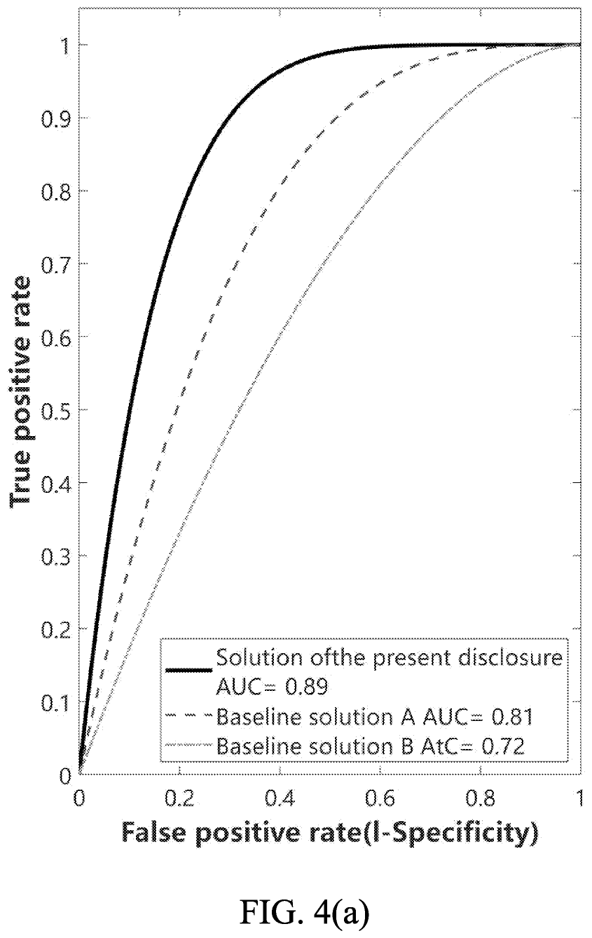

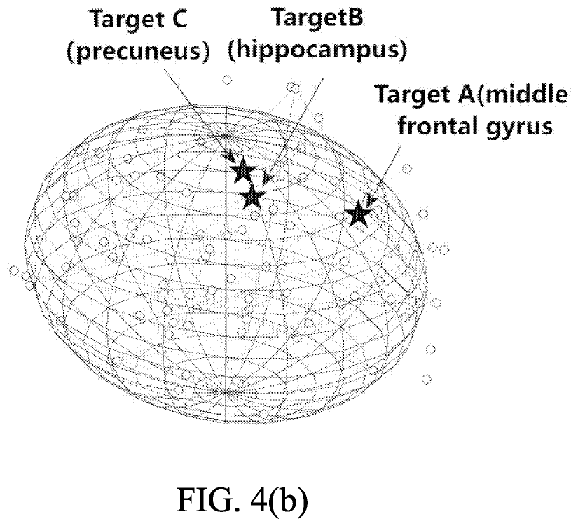

With reference to FIG. 4(a) and FIG. 4(b), an area under the curve (AUC) of the solution of the present disclosure on a validation set reaches 0.89, outperforming 0.72 for the traditional logistic regression and 0.81 for the static graph network. This data comparison confirms that incorporating a subjective psychological decompensatory tension and an underlying brain network temporal collapse process into a unified differentiable computational graph for joint representation can improve sensitivity of a system in capturing occult cognitive impairment risks.

While outputting a high-precision macroscopic prediction probability, the system generates microscopic intervention targets through the visualization and localization unit. By extracting a minimum value of the whole-node survival soft mask at a final simulation time step, and calculating a comprehensive risk index in combination with degree centrality feature values in the initial static brain network atlas, the system screens out top three hub brain region indices ranked by the risk index. The system inversely maps these three topological nodes into a standard Montreal Neurological Institute (MNI) three-dimensional brain atlas coordinate system. On a terminal display interface, these vulnerable brain regions, which first experience energy dissipation and physical compensation failure in the digital simulated surgery, are highlighted in color. This targeted distribution atlas, having physical causal interpretability, provides a direct spatial coordinate basis for a clinical anesthesia team to implement preventive brain protection interventions and focused regional oxygen saturation monitoring during actual surgical operations.

What is claimed is:

1. A perioperative vulnerable brain risk assessment system based on multimodal data, comprising:
a data acquisition and processing module configured to process multimodal clinical data to construct a subjective feature vector, an objective biochemical feature vector, a baseline state vector, and a static brain network atlas;
a network atlas reconstruction module configured to generate a perturbation matrix from the objective biochemical feature vector, apply the perturbation matrix to the static brain network atlas to obtain a reconstructed brain network atlas, and extract an objective physiological latent vector;
a vulnerability quotient quantification module configured to calculate a vulnerability reserve quotient based on a distance between a subjective psychological latent vector projected from the subjective feature vector and the objective physiological latent vector, in combination with a physiological tolerance threshold generated from the baseline state vector;
a cascading evolution simulation module configured to iteratively update an evolutionary adjacency matrix under the control of the vulnerability reserve quotient and a dynamic surgical stress input tensor generated from the baseline state vector, and extract a resilience evolution latent vector; and
a risk prediction and decision-making module configured to fuse the objective physiological latent vector, the subjective psychological latent vector, the resilience evolution latent vector, and the vulnerability reserve quotient to generate a comprehensive state representation vector, and output a risk probability and target node distribution spatial coordinates through network classification.

2. The perioperative vulnerable brain risk assessment system based on multimodal data according to claim 1, wherein the data acquisition and processing module comprises a subjective feature quantification unit, a biochemical feature smoothing unit, and a brain network atlas construction unit, wherein
the subjective feature quantification unit is configured to perform normalization on acquired subjective psychological scale scores by introducing an anti-zero-division smoothing term, and map scale score values of different dimensions into a unified numerical interval to generate a subjective feature vector;
the biochemical feature smoothing unit is configured to perform a logarithmic transformation with added smoothing parameters on blood biochemical indicator measurements to eliminate extreme value deviations caused by long-tail distributions, combine the transformed blood biochemical indicators to form an objective biochemical feature vector serving as a latent biochemical perturbation source within the system; and
the brain network atlas construction unit is configured to divide region of interest (ROI) nodes based on an automated anatomical labeling brain atlas to form a graph network node set, extract amplitude of low-frequency fluctuation (ALFF) values to form a node feature matrix, and calculate Pearson correlation coefficients for the time series of pairwise nodes to generate a static adjacency matrix according to an atlas connection threshold, thereby constructing the static brain network atlas through encapsulation.

3. The perioperative vulnerable brain risk assessment system based on multimodal data according to claim 1, wherein the network atlas reconstruction module comprises a biochemical perturbation generation unit and an edge weight reconstruction unit, wherein
the biochemical perturbation generation unit is configured to input a one-dimensional objective biochemical feature vector into a perturbation multi-layer perceptron model configured with a nonlinear activation function, extract a high-dimensional one-dimensional tensor from an output layer after feature dimensionality elevation in a hidden layer, and perform a tensor reshaping operation along a node ordering direction to construct a two-dimensional feature perturbation matrix with a dimension consistent with a dimension of the atlas; and
the edge weight reconstruction unit is configured to introduce a nonlinear gated Hadamard product mechanism containing learnable weights, and uses a nonlinear activation function to forcibly map the perturbation matrix and a bias matrix to generate a global gating mask within a predetermined numerical interval; and
perform an element-wise multiplication operation of elements at corresponding positions between the global gating mask and the static adjacency matrix, and modulate the static edge weights via attenuation gating using adaptively-adjusted disruption weights for different biochemical perturbation features, to generate the reconstructed brain network atlas.

4. The perioperative vulnerable brain risk assessment system based on multimodal data according to claim 3, wherein the network atlas reconstruction module further comprises a latent feature aggregation unit, wherein
the latent feature aggregation unit is configured to introduce a self-loop operation on the reconstructed adjacency matrix to construct an augmented matrix free of singularity risks and a diagonal degree matrix thereof, fuse node features through multi-layer graph convolution operations, extract a multi-dimensional feature tensor, smoothly compress the multi-dimensional feature tensor along the node dimension into a one-dimensional global representation using a global average pooling layer, and output the objective physiological latent vector.

5. The perioperative vulnerable brain risk assessment system based on multimodal data according to claim 1, wherein the vulnerability quotient quantification module comprises a latent space projection unit, a compensatory tension calculation unit, and a reserve quotient generation unit, wherein
the latent space projection unit is configured to, using a nonlinear projection network containing a batch normalization layer, project and map the subjective feature vector in a different feature dimension into a unified latent space with a dimension strictly consistent with a dimension of the objective physiological latent vector, to generate the subjective psychological latent vector;
the compensatory tension calculation unit is configured to calculate an aligned squared Euclidean distance between the subjective psychological latent vector and the objective physiological latent vector in the latent space, introduce an adaptive variance penalty smoothing mechanism during calculation to eliminate abnormal feature noise, and digitally quantify a subjective and objective latent decompensatory tension; and
the reserve quotient generation unit is configured to decode the baseline state vector constructed from basic physiological parameters of a patient using a feedforward perceptron network in combination with a soft activation function, generate an individualized physiological tolerance threshold, and when the aligned squared Euclidean distance exceeds the physiological tolerance threshold, output a positive difference using a rectified linear unit as a nonlinear truncation function to generate the vulnerability reserve quotient.

6. The perioperative vulnerable brain risk assessment system based on multimodal data according to claim 1, wherein the cascading evolution simulation module comprises a temporal stress generation unit and a node energy dissipation unit, wherein
the temporal stress generation unit is configured to, using a feedforward stress decoding network configured with positive constraints, in combination with a maximum simulation time step and a stress amplification factor, map and construct the baseline state vector into a dynamic surgical stress input tensor that monotonically increases over time steps;
the node energy dissipation unit is configured to extract a sum of edge weights of an adjacency matrix from a previous time step as a dynamic effective connectivity of nodes, take the reconstructed adjacency matrix as an initial evolution baseline, and normalize initial internal residual energy of all nodes to a fully loaded compensatory state; and
in an iterative simulation calculation, the node energy dissipation unit is configured to dynamically and controllably attenuate internal residual energy of each brain region node according to a vulnerability sensitivity multiplier modulated by the vulnerability reserve quotient, the dynamic surgical stress input tensor for the corresponding time step, and the dynamic effective connectivity containing an anti-zero-division smoothing term.

7. The perioperative vulnerable brain risk assessment system based on multimodal data according to claim 6, wherein the cascading evolution simulation module further comprises a topology dynamic update unit, wherein
the topology dynamic update unit is configured to, using a predetermined physical failure energy threshold as a truncation baseline, and employing a logistic function with a temperature hyperparameter, nonlinearly and smoothly map the internal residual energy of each brain region node into a continuously differentiable node survival soft mask within a predetermined numerical interval, to construct an end-to-end gradient backpropagation path;
perform a matrix outer product operation by extracting a column vector composed of whole-node survival soft masks to construct a global mask matrix, perform a Hadamard product operation with the adjacency matrix from the previous time step to trigger network cascading transfer, and dynamically update the evolutionary adjacency matrix; and
after reaching a predetermined maximum simulation time step, apply a graph readout mechanism to an extracted final evolutionary adjacency matrix, perform average pooling aggregation along a node feature dimension to compress local discrete states, and extract the resilience evolution latent vector containing dynamic collapse temporal information.

8. The perioperative vulnerable brain risk assessment system based on multimodal data according to claim 1, wherein the risk prediction and decision-making module comprises a feature fusion unit and a risk classification unit, wherein
the feature fusion unit is configured to perform a one-dimensional tensor concatenation operation along a feature axis on the objective physiological latent vector, the subjective psychological latent vector, the resilience evolution latent vector, and the scalar-dimensional vulnerability reserve quotient, to construct a comprehensive state representation vector with aligned feature dimensions; and
the risk classification unit is configured to input the comprehensive state representation vector into a fully connected feedforward classification network configured with a dropout mechanism, decode the comprehensive state representation vector via a normalized exponential function to generate a two-dimensional risk probability vector, and construct a cross-entropy loss function based on historical clinical follow-up data to perform joint optimization and parameter backpropagation updates; and
during an early warning phase, extract the probability of adverse neurocognitive outcomes exceeding an optimal decision threshold dynamically established by a receiver operating characteristic (ROC) curve of a validation set, and trigger a graded clinical warning in a terminal system.

9. The perioperative vulnerable brain risk assessment system based on multimodal data according to claim 8, wherein the risk prediction and decision-making module further comprises a visualization and localization unit, wherein
the visualization and localization unit is configured to extract a whole-node survival soft mask vector generated at a final simulation time step, perform descending order determination in combination with a topological degree centrality feature value representing a sum of direct physical connection edges in the static brain network atlas, and calculate and extract a predetermined number of target node indices with top-ranking comprehensive risk indices; and
based on a loaded standard medical imaging atlas template, inversely map the extracted target node indices one by one onto a three-dimensional brain atlas coordinate system, and render, anchor and output the target node distribution spatial coordinates on a structural MRI image interface of the patient.

10. The perioperative vulnerable brain risk assessment system based on multimodal data according to claim 8, wherein the process of performing joint optimization and parameter backpropagation updates by the risk classification unit specifically comprises:
the risk classification unit preprocesses historical clinical follow-up data into a two-dimensional one-hot encoding vector to construct a true label vector, and calculates a prediction error in conjunction with the two-dimensional risk probability vector;
the risk classification unit introduces a positive real number anti-overflow smoothing constant into the cross-entropy loss function to prevent a prediction probability from triggering an underlying computational graph crash; and
during the backpropagation process, the risk classification unit updates network weights using an adaptive moment estimation optimizer, and reversely transmits the prediction error in a backpropagation computational graph through a chain rule, thereby driving learnable parameters of a graph convolutional network, a multi-layer perceptron, and the evolutionary adjacency matrix in the preceding network atlas reconstruction module and cascading evolution simulation module to dynamically iterate towards minimizing the prediction error.
